(19) United States
(12) Patent Application Publication
(10) Pub. No.: US 2022/0280436 A1
(43) Pub. Date: Sep. 8, 2022

(54) SOFTSHELL CAPSULE FORMULATIONS, AND METHODS OF PREPARATION AND USE THEREOF

(71) Applicant: R.P. Scherer Technologies, LLC, Las Vegas, NV (US)

(72) Inventors: Jean Magloire Kameni, Lutz, FL (US); Linus Gomsi Fonkwe, Clearwater, FL (US); Ronak Savla, Fairfax, VA (US); Neftali Tosado, Cary, NC (US)

(21) Appl. No.: 17/629,126

(22) PCT Filed: Jul. 22, 2020
(86) PCT No.: PCT/US2020/042992

§ 371 Date: Jan. 21, 2022

Related U.S. Application Data
(60) Provisional application No. 62/877,580, filed on Jul. 23, 2019.

(51) Int. Cl. A61K 9/48

(52) U.S. Cl. CPC A61K 9/4816; A61K 9/4858

(57) ABSTRACT

Disclosed herein are softgel dosage forms, for example, stable aspirin softgel capsules. Also disclosed herein are methods of preparing such softgel dosage forms and methods of use thereof.

### FIELD

This disclosure relates to softshell capsule formulations, for example, free from animal derived products and starch. Also disclosed herein are methods of preparation of such softshell capsules and methods of use thereof.

### BACKGROUND

Encapsulating a solution or dispersion of a nutritional or pharmaceutical agent in a liquid carrier within a softshell capsule offers numerous advantages over other dosage forms such as compressed, coated or uncoated solid tablets or bulk liquid preparations. Such encapsulation of a solution or dispersion enables accurate delivery of a unit dose, which can be particularly important when relatively small amounts of active ingredient must be administered. Additionally, uniformity is more difficult to achieve with a tableting process, for example, where solids must be uniformly mixed and compressed, or the total dose of active ingredient must be incorporated into a bulk liquid carrier that must be measured out prior to each oral administration.

Moreover, soft capsules, most commonly, soft gelatin capsules, provide a dosage form which is more readily accepted by patients, since the capsules are easy to swallow and need not be flavored in order to mask any unpleasant taste of the active agent. Soft capsules are also more easily transported by patients than bulk liquids, since only the required number of doses need to be removed from the package.

Soft encapsulation of drugs further has the potential to improve bioavailability of pharmaceutical agents. Active ingredients are rapidly released in liquid form as soon as the shell ruptures. Complete disintegration of the capsule is not necessary for the active ingredients to become available for absorption, unlike the case of tableted compositions. Furthermore, relatively insoluble active ingredients can be dispersed in a liquid carrier to provide faster absorption.

However, gelatin-based soft capsules are not vegetarian, they have cross-linking potential and the variability of the raw material can be quite high. Vegicaps have been developed to provide a vegetarian form of capsules (i.e., cellulose-based shells) that provide some of the benefits of soft capsule shells. However, known vegicaps can be prone to damage during the encapsulation process, for example, vegicaps may burst, crack or deform during the tumble drying step and also require staging on one or more multi-layer conveyor belts for 5 min-40 min in order to withstand the impact force of the tumble dryer. Furthermore, the gel mass used to form the vegicaps has a maximum gel age of 14 days, while gelatin-based soft capsule shells have a maximum age of only 3 days. If such material is not used within the maximum age, then it must be discarded.

Accordingly, there is a need for improved softshell capsule formulations that are, for example, vegetarian and, in some embodiments, free of gelatin and/or starch (e.g., modified starch). Such softshell capsule formulations as described herein have high capsule performance and robustness during the encapsulation process, as compared to known vegicaps, and reduce processing time as compared to other capsule formulations.

### BRIEF SUMMARY

According to various embodiments, described herein is a softshell capsule formulation, comprising a synthetic polymer; a natural gelling agent; a buffering agent; a plasticizer; and water.

Also described herein are various embodiments of a method of preparing a softshell capsule formulation, comprising combining a synthetic polymer, a natural gelling agent, a buffering agent, a plasticizer and water to form a combination.

According to further embodiments, described herein is a method of using a softshell capsule formulation, comprising encapsulating a fill composition in the softshell capsule formulation.

### DETAILED DESCRIPTION

Described herein are various embodiments of softshell capsule formulations and methods of preparation and use thereof. It is to be understood that the invention is not limited to the details of construction or process steps set forth in the following description. The invention is capable of other embodiments and of being practiced or being carried out in a variety of ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “an active ingredient” includes a single active ingredient as well as a mixture of two or more different active ingredients.

As used herein, the term “about” in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term “about” includes the recited number±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Unless otherwise indicated, all parts and percentages are by weight. Weight percent (wt. %), if not otherwise indicated, is based on an entire composition free of any volatiles, that is, based on dry solids content.

Although the disclosure herein is with reference to particular embodiments, it is to be understood that these embodiments are merely illustrative of the principles and applications of the invention. It will be apparent to those skilled in the art that various modifications and variations can be made to the compositions and methods without departing from the spirit and scope of the invention. Thus, it is intended that the invention include modifications and variations that are within the scope of the appended claims and their equivalents.

### Softshell Capsule Formulations

Disclosed herein are softshell capsule formulations containing a synthetic polymer, a natural gelling agent, a buffering agent, a plasticizer and water. Softshell capsule formulations as described herein can be vegetarian and free of gelatin and/or a starch (e.g., modified starch). The softshell capsule formulations can be reprocessed and/or recycled such that any unused material (e.g., a gel mass of the softshell capsule formulation) can be formed into a netting and stored in a refrigerated environment (5° C. or lower) until further processing is desired. The softshell capsule formulations as described herein eliminate the gel mass hold time and the need of a staging conveyor during processing. For example, gelatin-based gel masses can only be held for 72 hours and vegicap gel masses can only be held for 14 days. Under such circumstances, these gel masses may have to be discarded if subsequent processing does not occur within these timeframes. In comparison, the gel mass form of the softshell capsule formulations as described herein can be held for more than 8 weeks, which reduces or eliminates the need to discard the material. Softshell capsule formulations as described herein also have less raw material variability and cross-linking potential as compared to other shell formulations. In embodiments, the ribbon thickness of softshell capsule formulations as described herein can be controlled to 0.018 in to 0.020 in whereas for gelatin capsules the ribbon thickness only could be controlled to 0.025 in to 0.040 in, which represents a 28% to 35% reduction for the softshell capsule formulations as described herein. Additionally, softshell capsule formulations as described herein have improved capsule performance and robustness during tumble drying as compared to traditional gelatin-based capsules and vegicaps. For example, gelatin capsules might experience twin/double defects and brittleness while vegicaps are relatively weak and cannot resist the impact force of a tumble dryer without a holding conveyor. Comparatively, softshell capsule formulations as described herein have strong seals and are very robust with good resilience from the chute and during tumble drying.

According to embodiments, the synthetic polymer contains at least one of a poly(N-vinyl lactam), povidone, crospovidone, a maleic anhydride copolymer, poly(2-ethyl-2-oxazoline), poly(ethyleneimine), polyurethane hydrogelsan acrylic acid polymer, a methacrylic acid polymer, methyl acrylate, ethyl acrylate, methyl methacrylate, ethyl methacrylate, aminoethyl acrylate, maleic anhydride, polymaleic acid, a polyacrylamide, poly(methacrylamide), poly(dimethylacrylamide), poly(N-isopropyl acrylamide), a polyolefinic alcohol, poly(N-vinyl caprolactam), a polyol, glycerol, polyglycerol, propylene glycol, polyoxyethylated sorbitol, polyoxyethylated glucose, a polyoxazoline, poly(methyloxazoline), poly(ethyloxazoline), a polyvinylamine, a polyvinylacetate, polyvinylacetate, polyvinyl acetate phthalate, a polyimine, polyethyleneimine, a polyurethane hydrogel, chitosan, a polysaccharide gum, zein, shellac, ammoniated shellac, shellac acetyl alcohol, shellac n-butyl stearate, esters thereof, homopolymers thereof, copolymers thereof, block copolymers thereof, graft copolymers thereof and/or combinations thereof. In embodiments, the synthetic polymer comprises povidone. In embodiments, the synthetic polymer is in an amount of about 10 wt % to about 50 wt %, or about 15 wt % to about 40 wt %, or about 20 wt % to about 30 wt %, or about 24 wt %, or about 25 wt %, or about 26 wt %, or about 27 wt %, or about 28 wt %, or about 29 wt %, or about 30 wt %, or about 31 wt %.

According to embodiments, the natural gelling agent includes at least one of carrageenan, xanthan gum, agar agar or pectin, sugar, sugar derived alcohol, starch, pregelatinized starch, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, polyethylene glycol, polyethylene oxide, polyvinyl alcohol, silicon dioxide, curdlan, furcelleran, egg white powder, lacto albumin, soy protein, chitosan and/or sodium laurel sulfate. In at least one embodiment, the natural gelling agent comprises carrageenan. The carrageenan can be at least one of iota carrageenan, kappa carrageenan and/or lambda carrageenan. In certain embodiments, the natural gelling agent is iota carrageenan. In embodiments, the natural gelling agent is in an amount of about 0.1 wt % to about 15 wt %, or about 0.5 wt % to about 14 wt %, or about 1 wt % to about 13 wt %, or about 2 wt % to about 12 wt %, or about 3 wt % to about 12 wt %, or about 4 wt % to about 11 wt %, or about 5 wt % to about 10 wt %, or about 6 wt % to about 9 wt %, or about 5 wt %, or about 5.5 wt %, or about 6.0 wt %, or about 6.5 wt %, or about 7.0 wt %, or about 7.5 wt %, or about 8.0 wt %, or about 8.5 wt %, or about 9.0 wt %.

According to embodiments, the buffer agent contains at least one of dibasic sodium phosphate, monobasic sodium phosphate, sodium bicarbonate, sodium citrate, disodium phosphate, calcium phosphate, dibasic calcium phosphate, tribasic calcium phosphate, monobasic potassium phosphate and/or dibasic potassium phosphate. In embodiments, the buffer agent comprises dibasic sodium phosphate. In embodiments, the buffering agent is in an amount of about 0.01 wt % to about 5 wt %, or about 0.05 wt % to about 4 wt %, or about 0.1 wt % to about 3 wt %, or about 0.5 wt % to about 3 wt %, or about 1.0 wt %.

According to various embodiments, the plasticizer contains at least one of glycerin, glycerol, adonitol, sorbitol, ribitol, galactitol, D-galactose, 1,3-dihydroxypropanol, glucose, sucrose, mannitol, xylitol, meso-erythritol, adipic acid, proline, hydroxyproline, polyol compound, monoglyceride, short- or medium-chain free fatty acid, monoacylglycerol ester, low molecular weight polymer, oligomer, copolymer, oil, small organic molecule, low molecular weight polyol having aliphatic hydroxyl, glycol ethers, poly(propylene glycol), multi-block polymer, single block polymer, low molecular weight poly(ethylene glycol), citrate ester-type, triacetin, propylene glycol, ethylene glycol, 1,2-butylene glycol, 2,3-butylene glycol, styrene glycol, diethylene glycol, triethylene glycol, tetraethylene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and/or allyl glycolate. In certain embodiments, the plasticizer is glycerin. In embodiments, the plasticizer is in an amount of about 10 wt % to about 30.0 wt %, or about 12 wt % to about 28 wt %, or about 15 wt % to about 25 wt %, or about 18 wt % to about 23 wt %, or about 17 wt %, or about 18 wt %, or about 19 wt %, or about 20 wt %, or about 21 wt %, or about 22 wt %, or about 23 wt %.

In certain embodiments, the softshell capsule formulation contains water. The water may be present in an amount of about 30 wt % to about 60 wt %, or about 35 wt % to about 55 wt %, or about 40 wt % to about 50 wt %, or about 42 wt %, or about 43 wt %, or about 44 wt %, or about 45 wt %, or about 45.5 wt %, or about 46 wt %, or about 47 wt %, or about 48 wt %. According to embodiments, a ratio of the water to the synthetic polymer is about 1:5 to about 5:1, or about 1:4 to about 4:1, or about 1:3 to about 3:1, or about 1:1, or about 2:1, or about 3:1, or about 4:1, or about 5:1.

In certain embodiments, the softshell capsule formulation is free of at least one of gelatin and/or starch. In embodiments, the softshell capsule formulation is alternatively, or additionally free of modified starch.

The softshell capsule formulation as described herein can have a shelf life of up to about 60 days. In embodiments, the softshell capsule formulation after drying has a water activity of about 0.150 Aw about 0.500 Aw, or about 0.166 Aw to about 0.350 Aw, or about 0.3473 Aw, or about 0.3297 Aw, or about 0.3196 Aw and a water activity of about 0.150 Aw about 0.500 Aw, or about 0.166 Aw to about 0.325 Aw.

The softshell capsule formulations as described herein may further include a fill composition. The fill composition may contain at least one of rapeseed oil, Labrasol® ALF (i.e., caprylocaproyl polyoxyl-8 glycerides or a nonionic water-dispersible surfactant for lipid-based formulations or a microemulsion comprising mono-, di- and triglycerides, polyethylene glycol-8, i.e., PEG-8 having a molecular weight 400 Da, mono- and diesters of caprylic and capric acids), medium chain triglyceride oil, polyethylene glycol and/or combinations thereof. Lipophilic and/or hydrophilic and/or alcohol fill compositions could also be encapsulated with the softshell capsule formulations as described herein.

In embodiments, the softshell capsule formulations contain rapeseed oil and have a burst strength of about 835 g to about 4,725 g and/or a seal thickness of about 0.0200 in to about 0.0250 in, or about 0.0234 in to about 0.0240 in. In embodiments, the softshell capsule formulations contain medium chain triglyceride oil and have a burst strength of about 985 g to about 6,000 g and/or a seal thickness of about 0.0200 in to about 0.0250 in, or about 0.0207 in to about 0.0247 in. In embodiments, the softshell capsule formulations contain polyethylene glycol and have a burst strength of about 1335 g to about 6,140 g and/or a seal thickness of about 0.0150 in to about 0.0250 in, about 0.0175 in to about 0.0231 in.

### Excipients

The softshell capsule formulations according to the disclosure can further include one or more pharmaceutically acceptable excipients. Examples of pharmaceutically acceptable excipients are described in the Handbook of Pharmaceutical Excipients, American Pharmaceutical Association (2012), which is incorporated by reference herein. Suitable excipients include, but are not limited to, colorants, lubricants, thermal lubricants, antioxidants, disintegrants, binding agents, diluents, glidants, anti-adherants, chelating agents, sweeteners, flavorants, surfactants, solubilizers, stabilizers, hydrophilic polymers, hydrophobic polymers, waxes, lipophilic materials, absorption enhancers, preservatives, cross-linking agents, bioadhesive polymers, pore formers and/or combinations thereof.

Examples of suitable binding agents include, but are not limited to, cellulosic polymers (e.g., hydroxypropylmethylcellulose, hydroxypropylcellulose, hydroxyethylcellulose, etc.), polyethylene glycol, an acrylic polymer, an acrylic copolymer, a graft copolymer of polyvinyl alcohol and polyethylene glycol, a polyvinyl alcohol, alginic acid, sodium alginate, starch, pregelatinized starch, sucrose, guar gum, salts thereof, derivatives thereof and combinations thereof. Additional binders include, but are not limited to, natural or synthetic waxes, fatty alcohols (e.g., lauryl, myristyl, stearyl, cetyl or cetostearyl alcohol), fatty acids, including, but not limited to, fatty acid esters, fatty acid glycerides (e.g., mono-, di-, and tri-glycerides), hydrogenated fats, hydrocarbons, stearic acid, hydrophobic and hydrophilic materials having hydrocarbon backbones, acacia, tragacanth, sucrose, gelatin, glucose, cellulose materials (e.g., methylcellulose and sodium carboxymethylcellulose (e.g., Tylose™)), magnesium aluminum silicate, polysaccharide acids, bentonites, polyvinylpyrrolidone (povidone), polymethacrylates, and/or pregelatinized starch (such as National™ 1511 and Starch 1500). Suitable waxes include, for example, beeswax, glycowax, castor wax, carnauba wax and/or other wax-like substances. A “wax-like” substance is defined as any material which is normally solid at room temperature and has a melting point of from about 30° C. to about 100° C.

Additional examples of binders which may be used include, but are not limited to, digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, natural and synthetic waxes and/or polyalkylene glycols. In certain embodiments, hydrocarbons having a melting point of between 25° C. and 90° C. may be included. Of the long-chain hydrocarbon binder materials, fatty (aliphatic) alcohols can be incorporated into the mixture according to certain embodiments. In further embodiments, the mixture or pharmaceutical composition may contain up to 80% (by weight) of at least one digestible, long chain hydrocarbon.

Examples of suitable disintegrants include, but are not limited to, sodium starch glycolate, clays (such as Veegum™ HV), celluloses (such as purified cellulose, methylcellulose, sodium carboxymethylcellulose, and carboxymethylcellulose), cross-linked sodium carboxymethylcellulose, starch, cross-linked polyvinylpyrrolidone (e.g., crospovidone), alginates, cornstarches and pre-gelatinized corn starches (such as National™ 1551 and National™ 1550), gums (such as agar, guar, locust bean, pectin, and tragacanth) and/or mixtures thereof. Disintegrants can be added at any suitable step during the preparation of the pharmaceutical compositions, such as prior to granulation or during a lubrication step prior to compression or encapsulation. The pharmaceutical compositions as described herein can include one or more disintegrants in the range of about 0.5% to about 30%, or about 1% to about 10%, or about 2% to about 6%, of the total weight of the formulation.

In at least one embodiment, the pharmaceutical composition includes a glidant. A glidant is an excipient that improves the flow characteristics of a compressible powder such as tablet ingredients and/or granules. Suitable glidants include, but are not limited to, silicon dioxide, colloidal silicon dioxide and/or combinations thereof.

Suitable diluents useful in pharmaceutical compositions as described herein include, but are not limited to, lactose (e.g., lactose (anhydrous), lactose (spray dried), lactose monohydrate), starch (e.g., directly compressible starch), mannitol, sorbitol, dextrose monohydrate, microcrystalline cellulose, dibasic calcium phosphate dihydrate, sucrose-based diluents, confectioner's sugar, monobasic calcium sulfate monohydrate, calcium sulfate dihydrate, calcium lactate trihydrate granular, dextrates (e.g., Emdex™), dextrose (e.g., Cerelose™), inositol, hydrolyzed cereal solids such as the Maltrons™ and Mor-Rex™ amylose, powdered cellulose (e.g., Elcema™), calcium carbonate, glycine, bentonite, polyvinylpyrrolidone, and/or combinations thereof. In certain embodiments, the pharmaceutical compositions described herein can include the diluents in the range of about 5% to about 99%, or from about 25% to about 90%, or from about 40% to about 80%, of the total weight of the formulation. Lactose has a melting point of about 202° C. Microcrystalline cellulose has a burning point of over 200° C. before it reaches a melting point, and is suitable as it does not have a low melting point.

Suitable lubricants include, but are not limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, silica, fumed silica, colloidal silica, calcium stearate, long chain fatty alcohols, boric acid, sodium benzoate and sodium acetate, sodium chloride, DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and/or combinations thereof. In certain embodiments, the pharmaceutical compositions may include one or more lubricants in an amount of from about 0.1% to about 10%, or from about 0.2% to about 8%, or from about 0.25% to about 5%, of the total weight of the formulation. Magnesium stearate is a lubricant suitable for use in certain embodiments of the pharmaceutical compositions. Magnesium stearate has a melting point of about 90° C. Although magnesium stearate has a low melting point, it can be utilized in small amounts (e.g., about 0.5%) as a lubricant without significantly affecting the stability of the peripheral opioid formulations according to embodiments herein.

Suitable anti-adherents include, but are not limited to, talc, cornstarch, colloidal silicone dioxide (Cab-O-Sil™), DL-Leucine, sodium lauryl sulfate and/or metallic stearates. In certain embodiments, the pharmaceutical compositions can include an anti-adherent in an amount from about 0.1% to about 15%, or from about 0.25% to about 10%, or from about 0.5% to about 5%, of the total weight of the formulation. Colloidal silicon dioxide is an anti-adherent agent suitable for use in some embodiments of the pharmaceutical compositions in an amount from about 0.1% to about 10%, or from about 0.25% to about 5%, or from about 0.5% to about 2%, of the total weight of the formulation. Colloidal silicon dioxide has a melting point of about 1700° C.

Other excipients (such as colorants, flavorant and sweeteners) can be utilized in embodiments of the pharmaceutical compositions where they impart little to no deleterious effect on the stability of the pharmaceutical composition.

According to embodiments, the softshell capsule composition contains at least one of a colorant, opacifier, flavorant, sweetener, preservative, embrittlement inhibiting agent and/or disintegrant. In embodiments, the colorant contains at least one of an azo dye, quinophthalone dye, triphenylmethane dye, xanthene dye, iron oxide, iron hydroxide, titanium dioxide, sunset yellow, allura red, amaranth, koki neil red, azogeranin, tartrazine, brilliant black, canthaxanthin, patent blue, fast green, brilliant blue, acid green, erythrosine, quinoline yellow, indigotin, curcumin, carbon black and/or combinations thereof. In embodiments, the opacifier contains titanium dioxide. According to embodiments, the flavorant contains at least one of a natural flavor oil, an artificial flavor oil, a synthetic flavor oil, a flavoring aromatic, a flavoring oils, an oleoresin, plant extract, leaf extract, flower extract, fruit extract, spearmint oil, peppermint oil, eucalyptus oil, nutmeg oil, allspice oil, mace, almond oil, menthol oil, citrus oil, lemon oil, orange oil, lime oil, grapefruit oil and/or combinations thereof. In embodiments, the sweetener contains at least one of agave syrup, stevia, erythritol, xylitol, sorbitol, yacon syrup, aspartame, saccharin, cyclamate, sucralose, monk fruit extract and/or combinations thereof. The preservative contains at least one of a methylparaben, propylparaben, sodium methylhydroxybenzoate, sodium ethylhydroxybenzoate, sodium butyhydroxybenzoate, a quaternary ammonium compound, benzalkonium chloride and/or combinations thereof. In embodiments, the embrittlement inhibiting agent contains at least one of sorbitol, sorbitans, polyhydric alcohols and/or combinations thereof. In embodiments, the disintegrant contains at least one of polyvinylpyrrolidone, croscarmellose sodium, sodium starch glycolate and/or combinations thereof.

### Methods of Preparing the Dosage Forms

Disclosed herein are methods of preparing a softshell capsule formulation. In embodiments, the methods include combining a synthetic polymer, a natural gelling agent, a buffering agent, a plasticizer and water to form a combination. In embodiments, the methods include premixing a synthetic polymer (including colorants), dissolving a buffer agent in water inside a melter, premixing a natural gelling agent and a plasticizer in a separate container, and transferring the premixture of natural gelling agent and plasticizer into the melter. These methods can further include heating the combination to form a molten mass. In embodiments, the molten mass is a uniform molten mass. The method may further include extruding the molten mass to form ribbons. Additionally, the method may include casting the ribbons on drums and forming soft capsule shells using a rotary die encapsulation apparatus. In embodiments, the ribbons may have a thickness of about 0.001 in to about 0.050 in, or about 0.005 in to about 0.030 in, or about 0.010 in to about 0.025 in, or about 0.015 in to about 0.021 in, or about 0.017 in, or about 0.018 in, or about 0.019 in, or about 0.020 in, or about 0.021 in, or about 0.022 in. In embodiments, a netting can be formed from the combination. The netting can be subsequently melted and reused to form ribbons.

The combining may further include mixing the plasticizer with the water to form a plasticizer solution and mixing the synthetic polymer, natural gelling agent and/or buffering agent with the plasticizer solution. The combining may further include mixing the plasticizer with the natural gelling agent to form a solution, mixing the solution with water to form a plasticizer solution and mixing the synthetic polymer and buffering agent with the plasticizer solution. In embodiments, the combining comprises introducing each of the synthetic polymer, natural gelling agent, buffering agent, plasticizer and water into a low or high shear mixer. In embodiments, the combining can be for about 1 min to about 3 hours, or about 5 min to about 2.5 hours, or about 15 min to about 2.0 hours, or about 20 min to about 1.5 hours, or about 30 min to 1.0 hour, or about 5 min to about 30 min. The combining can be at a temperature of about 45° C. to about 90° C., or about 50° C. to about 85° C., or about 55° C. to about 80° C., or about 60° C. to about 70° C., or about 55° C., or about 60° C., or about 65° C. The combining can further include increasing the temperature to about 95° C. to about 125° C., or about 100° C. to about 120° C., or about 105° C. to about 115° C., or about 95° C., or about 96° C., or about 97° C., or about 98° C., or about 99° C., or about 100° C.

According to embodiments, the method may further include transferring the combination to a receiving tank. The combination, or material in the receiving tank, may be transferred to a heated vessel to heat the material therein. In embodiments, the receiving tank together with the material therein, may be transferred to a drum unloader. In the drum unloader, a heating platen or melting plate is lowered onto the top surface of the material (i.e., the combination or shell mass) in the receiving tank. In a melt-on-demand process, the platen is configured to heat the material in the receiving tank to a temperature of about 70° C. to about 110° C., or about 90° C. upon receipt of a control signal from an encapsulation machine, and/or from a heated intermediate storage vessel, indicating that more molten gel mass is required. Using heat, the platen transforms the material in the receiving tank into the molten gel mass, which can be subsequently transferred (e.g., pumped) to the film-forming extrusion system of an encapsulation machine (or to the heated intermediate storage vessel). When the predetermined level of molten mass in the heated intermediate storage vessel or in the encapsulation machine is reached, another control signal is sent to the drum unloader to stop the melt and transfer process. Once formed, the molten gel mass is suitable for further processing including color addition). In embodiments, the molten gel mass may be pumped from a drum unloader into a heated intermediate storage vessel. The heated vessel may heat the combination to a temperature of about 80° C. to about 115° C., or about 85° C. to about 100° C., or about 88° C. to about 95° C. According to embodiments, the method may include injecting a coloring agent into the combination.

The method can further include transferring the combination to an encapsulation apparatus. In embodiments, the method includes encapsulating a fill material within a softshell capsule formed from the combination to form a plurality of softshell capsule dosage forms. The method can further include drying the plurality of softshell capsule dosage forms in a tumble dryer and a drying tunnel. Softshell capsule dosage forms may be placed in a drying tunnel after tumble drying for secondary drying until the capsules are fully dried. Embodiments can additionally include packaging the plurality of softshell capsule dosage forms.

### Methods of Using the Dosage Forms

According to various embodiments, disclosed herein are methods of using a softshell capsule formulation. The methods can include encapsulating a fill composition within the softshell capsule formulation. In embodiments, the fill composition comprises at least one of a carrier, a vitamin, an anti-inflammatory or a nutritional, cosmetic or pharmaceutical agent in a liquid carrier or in the form of a solution or dispersion. In embodiments, the encapsulating can be by a method described above including heating the ingredients to form a molten mass and forming ribbons over a drum. The softshell capsule formulations describe herein can encapsulate materials using other encapsulation methods, apparatus and techniques known to those of ordinary skill in the art.

### EXAMPLES

### Example 1—Comparison of Softshell Capsule Formulations to Vegicap-Based Formulations

Vegicap-based soft capsule shells and softshell capsule formulations as described herein were prepared. The vegicap-based soft capsule shells were from R.P. Scherer Technologies' a.k.a. OptiShell®. The softshell capsule formulations had the ingredients at the corresponding amounts as shown in Table 1.

TABLE 1
Softshell Capsule Formulations according to Invention

| Ingredient | Quantity (wt %) |
|---|---|
| Iota Carrageenan | 7.5 |
| Kollidon K30 | 26.0 |
| (povidone) | |
| Na Phosphate dibasic | 1.0 |
| Glycerin | 20.0 |
| Water | 45.5 |
| Total: | 100 |

The softshell capsule formulations were evaluated for their fill material compatibility. The results are presented in Table 2. The softshell capsule formulations were compatible with all fill materials tested, that is, they were compatible with lipophilic and hydrophilic fills.

TABLE 2
Softshell Capsule Formulations Fill Material Compatibility

| | Reaction | Compatibility |
|---|---|---|
| Fill Material | (Y/N) | (Y/N) |
| 90% PEG 400 + 10% Polysorb | N | Y |
| Capmul MCM Oil | N | Y |
| Peanut Oil NF | N | Y |
| Coconut Oil | N | Y |
| Soybean Oil | N | Y |

The softshell capsule formulations also were evaluated for their burst strength. The formulations were processed using a standard encapsulation process used for the OptiShell® capsules. The burst strength results are set forth in Table 3. As a result of the burst strength tests, it was determined that a holding conveyor belt was not needed as is required for the OptiShell® formulations. Eliminating the holding conveyor belt step can potentially eliminate about 5 min to about 40 min of cycle time per batch resulting in improved process efficiency. The burst strength results for the softshell capsule formulations were compared to the burst strength results for OptiShell® compositions (also shown in Table 3). Softshell formulations according to the present invention had higher burst strength values than the OptiShell® capsules.

TABLE 3
Burst Strength Results (Comparative)

| | | | |
|---|---|---|---|
| Average | Minimum | Maximum | Standard |
| (g) | (g) | (g) | Deviation |

OptiShell ®

| | | | |
|---|---|---|---|
| 1110 | 695 | 1921 | 334 |
| 1327 | 681 | 2654 | 245 |
| 1263 | 726 | 2643 | 323 |
| 1257 | 509 | 2354 | 327 |
| 1253 | 503 | 3292 | 456 |
| 1095 | 667 | 2051 | 248 |
| 1544 | 599 | 2816 | 452 |
| 1417 | 557 | 2474 | 351 |

Softshell Capsule Formulations (Inventive)

| | | | |
|---|---|---|---|
| 1903 | 836 | 4724 | 773 |
| 2484 | 989 | 5093 | 1076 |
| 2422 | 1337 | 6137 | 945 |

The water activity for dried softshell capsule formulations according to the invention was compared with the water activity for the OptiShell® formulations. The results are shown in Table 4. The softshell capsule formulations as described herein had less water activity and no leakers as compared to the OptiShell® formulations.

TABLE 4
Water Activity Results (Comparative)

| | | |
|---|---|---|
| Drying Time | Water Activity | Number of |
| (hours) | (Aw) | Leakers |

OptiShell ®

| | | |
|---|---|---|
| 48 | 0.464 | 7 |
| 48 | 0.456 | 7 |
| 45 | 0.436 | 0 |
| 44 | 0.454 | 4 |
| 72 | 0.409 | 0 |
| 72 | 0.450 | 0 |
| 92 | 0.377 | 2 |
| 94 | 0.386 | 41 |

Softshell Capsule Formulations (Inventive)

| | | |
|---|---|---|
| 4 | 0.325 | 0 |
| 16.5 | 0.217 | 0 |
| 28 | 0.175 | 0 |
| 43.5 | 0.166 | 0 |
| 67 | 0.207 | 0 |

The softshell capsule formulations were evaluated for stability over a nine (9) month period. The results are presented in Table 5. The softshell capsule formulations according to the invention were stable over the nine (9) month period.

TABLE 5
Stability Results for Softshell Capsule Formulations

| Tests | Results 1 | Results 2 | Results 3 |
|---|---|---|---|
| Condition | 25° C. | 30 C. | 40° C. |
| | @ 60% R.H. | @ 65 R.H. | @ 75% R.H. |
| Water Content | 0.0096% | 0.0467% | 0.0374% |
| Disintegration | 10 min (Average) | 10 min (Average) | 10 min (Average) |
| Hardness | 1.9N (Average) | 1.7N (Average) | 1.9N (Average) |
| Water Activity | 0.3473 Aw | 0.3196 Aw | 0.3297 Aw |

### Example 2—Preparation of Softshell Gel Mass and Encapsulation of Fill Composition

A 250 kg gel mass was prepared having the ingredients as shown in Table 6. The gel mass was prepared according to the method set forth in Table 7. Fill composition were prepared having the components as set forth in Tables 8 and 9. The prepared gel mass was then fed to an encapsulation apparatus for encapsulating the fill compositions.

TABLE 6
Shell Mass Composition

| | | Theoretical Weight | |
|---|---|---|---|
| Item Description | %, w/w | Per Batch | |
| Povidone K30 | 26.0 | 65.00 | kg |
| Iota Carrageenan, NF | 7.5 | 18.75 | kg |
| Sodium Phosphate, Dibasic | 1.0 | 2.50 | kg |
| Glycerin | 20.0 | 50.00 | kg |
| Water (A) | 2.5 | 6.25 | kg |
| Water (B) | 43 | 107.50 | kg |

| Total Theoretical | |
|---|---|
| Weight Per Batch (kg) | |
| 250.0 | kg |

TABLE 7
Shell Mass Preparation Method
Instructions

| | |
|---|---|
| 1 | Set the temperature on the melter to about 50° C. to about 100° C., or about 60° C. to |
| | about 80° C. Draw a vacuum on the melter at about −1500 millibars to about −500 |
| | millibars, or about −1200 millibars to about −800 millibars, and ensure the valve is |
| | closed. Obtain full vacuum on the melter. Once full vacuum has been reached close the vacuum valve. |
| 2 | Vacuum transfer approximately three-quarters (¾) of the water (B) into the melter. |
| | Reserve one-quarter (¼) of the water to use for flushing the vacuum transfer line |
| | after the addition of Glycerin plasticizer (Step 6), and the Povidone K30 and Iota |
| | Carrageenan (Step 10). |
| 3 | Vacuum transfer Sodium Phosphate, Dibasic. Slowly transfer through the bottom |
| | inlet using a hose attached to a dip tube, the Water and Sodium Phosphate, Dibasic. |
| 4 | Run the homogenizer at about 500 RPM to about 1500 RPM, or about 900 RPM to about 1100 RPM. |
| 5 | Mix for at least about 10 minutes, at least about 15 minutes, at least about 20 |
| | minutes, at least about 25 minutes, at least about 30 minutes, at least about 45 |
| | minutes or for about 10 minutes to about 120 minutes until completely dissolved. |
| 6 | Add item 1 to item 2: |
| | 1. Water |
| | 2. Glycerin |
| | Using a dip tube, coarsely blend the two liquids together. Slowly transfer through |
| | the bottom inlet, using the hose attached to the dip tube, the Glycerin and Water. |
| | Use about half (½) of the water reserved from step 2 to flush the vacuum transfer line. |
| 7 | Set an anchor sweep to about 10 RPM to about 50 RPM, about 15 RPM to about 40 |
| | RPM, 20 RPM to about 30 RPM, or 23 RPM to about 27 RPM and the emulsifier |
| | stirrer to about 250 RPM to about 750 RPM, about 300 RPM to about 600 RPM, |
| | about 350 RPM to about 55) RPM, or about 470 RPM to about 530 RPM. |
| 8 | Obtain full vacuum of about −1500 millibars to about −500 millibars, or about −1200 |
| | millibars to about −800 millibars on the melter. Close the vacuum valve. |
| 9 | When the temperature of the liquid in the melter reaches about 50° C. to about 100° C., |
| | or about 60° C. to about 80° C., or about 55° C. to about 60° C., turn off the homogenizer. |
| 10 | Vacuum transfer: |
| | Povidone K30 |
| | Iota Carrageenan, NF |
| | Slowly transfer through the bottom inlet, using a 1.5″ PVC beverage grade hose, the |
| | Povidone K30 and Iota Carrageenan. Use the remainder of the water reserved from |
| | step 2 to flush vacuum the transfer line. |
| 11 | Obtain a full vacuum of about −1500 millibars to about −500 millibars, |
| | or about −1200 millibars to about −800 millibars on the melter. |
| 12 | Close the vacuum valve. |
| 13 | Allow the slurry to mix for about 10 minutes to about 120 minutes until completely dissolved. |
| 14 | Set the temperature on the melter to about 50° C. to about 125° C., about 60° C. to about |
| | 120° C., or about 75° C. to about 110° C., or about 80° C. to about 100° C. |
| 15 | When the temperature of the shell mass reaches about 75° C. to about 110° C., about |
| | 80° C. to about 102° C., or about 86° C. to about 90° C., pull vacuum for about 10 seconds |
| | to about 90 seconds, about 15 seconds to about 60 seconds, about 20 seconds to |
| | about 45 seconds, or about 25 seconds to about 30 seconds. Close the vacuum valve. |
| 16 | Hold the melt for at least about 30 minutes to about 360 minutes, about 45 minutes |
| | to about 240 minutes, or about 60 minutes to about 180 minutes. |
| 17 | Pull vacuum for about 10 seconds to about 90 seconds, about 15 seconds to about 60 |
| | seconds, about 20 seconds to about 45 seconds, or about 25 seconds to about 30 seconds. |
| 18 | Stop the mixer and release the vacuum |
| 19 | Pressurize the vessel to about 250 mbar to about 1250 mbar, about 500 mbar to |
| | about 1000 mbar, or about 750 mbar to about 1000 mbar. |
| 20 | Record the finish time. |
| 21 | Carefully discharge the molten shell mass into one or more pre-weighed tank. |

TABLE 8
Fill Composition (Glycerin)
Master Formula
Theoretical Lot quantity = 15,000 softgels

| | | | Theoretical |
|---|---|---|---|
| Milligrams | % | | Weight |
| Per Softgel | w/w | Item Description | Per Batch |
| 100 | 100 | GLYCERIN ANHYDROUS | 1500.00 g |
| Total | | | Total Theoretical |
| | | | Weight Per Batch |
| 100.00 | 100.00 | | 1,500.00 g |

TABLE 10
Fill Composition (Labrasol)
Master Formula
Theoretical quantity = 15,000 softgels

| | | | Theoretical |
|---|---|---|---|
| Milligrams | % | | Weight |
| Per Softgel | w/w | Item Description | Per Batch |
| 100 | 100 | Labrasol ALF | 1500.00 g |
| Total | | | Total Theoretical |
| | | | Weight Per Batch |
| 100.00 | 100.00 | | 1,500.00 g |

TABLE 11
Encapsulation Apparatus Parameters

| | | Encap- | | Fill | Theoretical |
|---|---|---|---|---|---|
| | Fill | sulation | Ribbon | Weight | Qty |
| Sublot | Material | HZ | (inch) | (g) | (softgels) |
| A | Glycerin | 3 | 0.025-0.040 | 0.100 | 15000 |
| B | Labrasol | 3 | 0.025-0.040 | 0.100 | 15000 |

The softshell capsules formed from the gel mass having the 100% Labrasol ALF fill, were encapsulated using two different dies. Table 12 provides the hardness data points collected.

TABLE 12
Hardness Data for the Labrasol ALF Capsules

| Test Date | A | B |
|---|---|---|
| 19MC-113B | 3.6 | 3.5 |
| | 3.8 | 3.3 |
| | 3.4 | 3.4 |
| | 3.5 | 3.3 |
| | 3.8 | 3.2 |
| Average | 3.62 | 3.34 |
| 19MC-113B | 4.8 | 4.7 |
| | 4.5 | 4.4 |
| | 5 | 4.6 |
| | 4.9 | 4.5 |
| | 4.9 | 4.5 |
| Average | 4.82 | 4.54 |

The preceding description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail in order to avoid unnecessarily obscuring the present invention. Thus, the specific details set forth are exemplary. Particular embodiments may vary from these exemplary details and still be contemplated to be within the scope of the present invention.

Although the operations of the methods herein are described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the invention should, therefore, be determined with reference to the appended claims, along with the full scope of equivalents to which such claims are entitled.

I/We claim:

1. A softshell capsule formulation, comprising:
a synthetic polymer;
a natural gelling agent;
a buffering agent;
a plasticizer; and
water.

2. The softshell capsule formulation of claim 1, wherein the synthetic polymer comprises at least one of a poly(N-vinyl lactam), povidone, crospovidone, a maleic anhydride copolymer, poly(2-ethyl-2-oxazoline), poly(ethyleneimine), polyurethane hydrogelsan acrylic acid polymer, a methacrylic acid polymer, methyl acrylate, ethyl acrylate, methyl methacrylate, ethyl methacrylate, aminoethyl acrylate, maleic anhydride, polymaleic acid, a polyacrylamide, poly(methacrylamide), poly(dimethylacrylamide), poly(N-isopropyl acrylamide), a polyolefinic alcohol, poly(N-vinyl caprolactam), a polyol, glycerol, polyglycerol, propylene glycol, polyoxyethylated sorbitol, polyoxyethylated glucose, a polyoxazoline, poly(methyloxazoline), poly(ethyloxazoline), a polyvinylamine, a polyvinylacetate, polyvinylacetate, polyvinyl acetate phthalate, a polyimine, polyethyleneimine, a polyurethane hydrogel, chitosan, a polysaccharide gum, zein, shellac, ammoniated shellac, shellac acetyl alcohol, shellac n-butyl stearate, esters thereof, homopolymers thereof, copolymers thereof, block copolymers thereof, graft copolymers thereof and combinations thereof.

3. (canceled)

4. The softshell capsule formulation of claim 1, wherein the natural gelling agent comprises at least one of carrageenan, xanthan gum, agar agar or pectin, sugar, sugar derived alcohol, starch, pregelatinized starch, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, polyethylene glycol, polyethylene oxide, polyvinyl alcohol, silicon dioxide, curdlan, furcelleran, egg white powder, lacto albumin, soy protein, chitosan and sodium laurel sulfate.

5. (canceled)

6. The softshell capsule formulation of claim 4, wherein the carrageenan comprises at least one of iota carrageenan, kappa carrageenan and lambda carrageenan.

7. (canceled)

8. The softshell capsule formulation of claim 1, wherein the buffering agent comprises at least one of dibasic sodium phosphate, monobasic sodium phosphate, sodium bicarbonate, sodium citrate, disodium phosphate, calcium phosphate, dibasic calcium phosphate, tribasic calcium phosphate, monobasic potassium phosphate and dibasic potassium phosphate.

9. (canceled)

10. The softshell capsule formulation of claim 1, wherein the plasticizer comprises at least one of glycerin, glycerol, adonitol, sorbitol, sorbitol blend, ribitol, galactitol, D-galactose, 1,3-dihydroxypropanol, glucose, sucrose, mannitol, xylitol, meso-erythritol, adipic acid, proline, hydroxyproline, polyol compound, monoglyceride, short- or medium-chain free fatty acid, monoacylglycerol ester, low molecular weight polymer, oligomer, copolymer, oil, small organic molecule, low molecular weight polyol having aliphatic hydroxyl, glycol ethers, poly(propylene glycol), multi-block polymer, single block polymer, low molecular weight poly(ethylene glycol), citrate ester-type, triacetin, propylene glycol, ethylene glycol, 1,2-butylene glycol, 2,3-butylene glycol, styrene glycol, diethylene glycol, triethylene glycol, tetraethylene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and allyl glycolate.

11. (canceled)

12. (canceled)

13. The softshell capsule formulation of claim 1, further comprising a colorant, wherein the colorant comprises at least one of an azo dye, quinophthalone dye, triphenylmethane dye, xanthene dye, iron oxide, iron hydroxide, titanium dioxide, sunset yellow, allura red, amaranth, koki neil red, azogeranin, tartrazine, brilliant black, canthaxanthin, patent blue, fast green, brilliant blue, acid green, erythrosine, quinoline yellow, indigotin, curcumin, carbon black and combinations thereof.

14. The softshell formulation of claim 1, further comprising an opacifier, wherein the opacifier comprises titanium dioxide.

15. The softshell capsule formulation of claim 1, further comprising a flavorant, wherein the flavorant comprises at least one of a natural flavor oil, an artificial flavor oil, a synthetic flavor oil, a flavoring aromatic, a flavoring oils, an oleoresin, plant extract, leaf extract, flower extract, fruit extract, spearmint oil, peppermint oil, eucalyptus oil, nutmeg oil, allspice oil, mace, almond oil, menthol oil, citrus oil, lemon oil, orange oil, lime oil, grapefruit oil and combinations thereof.

16. (canceled)

17. The softshell capsule formulation of claim 1, further comprising a preservative, wherein the preservative comprises at least one of a methylparaben, propylparaben, sodium methylhydroxybenzoate, sodium ethylhydroxybenzoate, sodium butyhydroxybenzoate, a quaternary ammonium compound, benzalkonium chloride and combinations thereof.

18. The softshell capsule formulation of claim 1, further comprising an embrittlement inhibiting agent, wherein the embrittlement inhibiting agent comprises at least one of sorbitol, sorbitans, polyhydric alcohols and combinations thereof.

19. The softshell capsule formulation of claim 1, further comprising a disintegrant, wherein the disintegrant comprises at least one of polyvinylpyrrolidone, croscarmellose sodium, sodium starch glycolate and combinations thereof.

20-24. (canceled)

25. The softshell capsule formulation of claim 1, wherein a ratio of the water to the synthetic polymer is about 1:5 to about 5:1.

26. The softshell capsule formulation of claim 1, wherein the softshell capsule formulation is free of at least one of gelatin, starch or modified starch.

27. (canceled)

28. The softshell capsule formulation of claim 1, comprising at least one of a gel mass shelf life of up to about 60 days or a shelf life of about 60 days.

29. (canceled)

30. (canceled)

31. The softshell capsule formulation of claim 1 further comprising a fill composition, wherein the fill composition comprises at least one of rapeseed oil, medium chain triglyceride oil, polyethylene glycol and combinations thereof.

32. The softshell capsule formulation of claim 31, comprising the rapeseed oil and having at least one of a burst strength of about 835 g to about 4,725 g or a seal thickness of about 0.0150 in to about 0.0250 in, about 0.0175 in to about 0.0231 in.

33. The softshell capsule formulation of claim 31, comprising the medium chain triglyceride oil and having at least one of a burst strength of about 985 g to about 6,000 g or a seal thickness of about 0.0200 in to about 0.0250 in, or about 0.0207 in to about 0.0247 in.

34. The softshell capsule formulation of claim 31, comprising the polyethylene glycol and having a burst strength of about 1335 g to about 6,140 g.

35-37. (canceled)

38. The softshell capsule formulation of claim 1, wherein the softshell capsule formulation has a water activity of about 0.150 Aw about 0.500 Aw.

39-105. (canceled)
